(19) United States
(12) Patent Application Publication
(10) Pub. No.: US 2023/0016252 A1
(43) Pub. Date: Jan. 19, 2023

(54) Cassette for Sterilizing Curing Light Devices within an Autoclave

(71) Applicant: Michael Gennaro, Henderson, NV (US)

(72) Inventor: Michael Gennaro, Henderson, NV (US)

(21) Appl. No.: 17/376,051

(22) Filed: Jul. 14, 2021

(51) Int. Cl. A61L 2/07; A61L 2/26

(52) U.S. Cl. CPC A61L 2/07; A61L 2/26

(57) ABSTRACT

An article of manufacture for providing a cassette to sterilize curing light devices within an autoclave is disclosed. The cassette includes an enclosing cassette base having a base surface, sidewalls, and an open top, a saddle for restraining a curing light device, and a plurality of venting openings in the base surface. The cassette is made of a cassette material permitting use in the autoclave having steam when in operation and is capable of holding up to 3 saddles for different curing light devices.

### TECHNICAL FIELD

This application relates in general to an article of manufacture for providing a healthcare sterilization device, and more specifically, to an article of manufacture for providing a cassette to sterilize curing light devices within an autoclave.

### BACKGROUND

Dental practices use curing light devices to emit light at a frequency that causes various dental substances to solidify and cure into a repair to one or more teeth within a patient's mouth. The curing light devices must be sterilized between uses with different patients to prevent possible spread of viruses and bacteria that can cause illness in these patients. Once the curing light devices are sterilized, the devices must be securely sealed within an isolating package to prevent the devices from being contaminated before use with a subsequent patient.

Most dental instruments are sterilized between uses by placing the devices within an autoclave to expose the instruments to steam sufficient to eliminate any pathogens present on the instruments from a previous patient. The curing light devices are fragile devices that usually are sterilized by shipping the device to a processing facility since the curing light devices typically cannot survive undamaged when cleaned within an autoclave with other dental instruments. This requirement to send the curing light devices to a processing facility for sterilization requires a dental practice to utilize additional devices while previously used devices are transported, cleaned, and returned. This sterilization process also adds additional expenses to the dental practice that are imposed upon patients in the required fees to obtain treatment from the dental practice.

Therefore, a need exists for an article of manufacture for providing a cassette to sterilize curing light devices within an autoclave. The present invention attempts to address the limitations and deficiencies in prior solutions according to the principles and example embodiments disclosed herein.

### SUMMARY

In accordance with the present invention, the above and other problems are solved by providing an article of manufacture for a cassette to sterilize curing light devices within an autoclave according to the principles and example embodiments disclosed herein.

In one embodiment, the present invention is an article of manufacture for providing a cassette to sterilize curing light devices within an autoclave. The cassette includes an enclosing cassette base having a base surface, sidewalls, and an open top, a saddle for restraining a curing light device, and a plurality of venting openings in the base surface.

In another aspect of the present invention, the cassette is made of a cassette material permitting use in the autoclave having steam when in operation and is capable of holding up to 3 saddles for different curing light devices.

In another aspect of the present invention, the cassette material is plastic.

In another aspect of the present invention, the saddle is made of polythene.

In another aspect of the present invention, the saddle is securing to the cassette base surface using a securing device.

In another aspect of the present invention, the securing device comprises stainless steel rivets, screws, and nuts and bolt pairs.

In another aspect of the present invention, the cassette has a plurality of saddles.

In another aspect of the present invention, the plurality of saddles includes individual saddles of different sizes holding different curing light devices.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment of an article of manufacture for providing a cassette to sterilize curing light devices within an autoclave according to the present invention.

FIGS. 2a-c illustrate multiple views of an article of manufacture for providing a cassette to sterilize curing light devices within an autoclave according to the present invention.

### DETAILED DESCRIPTION

This application relates in general to an article of manufacture for providing a healthcare sterilization device, and more specifically, to an article of manufacture for providing a cassette to sterilize curing light devices within an autoclave according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

The terms “healthcare worker” and “user” refer to an entity, e.g., a human, using an article of manufacture for providing a cassette to sterilize curing light devices within an autoclave associated with the invention. The term user herein refers to one or more users.

The term “invention” or “present invention” refers to the invention being applied for via the patent application with the title “Cassette for Sterilizing Curing Light Devices within an Autoclave.” Invention may be used interchangeably with cassette.

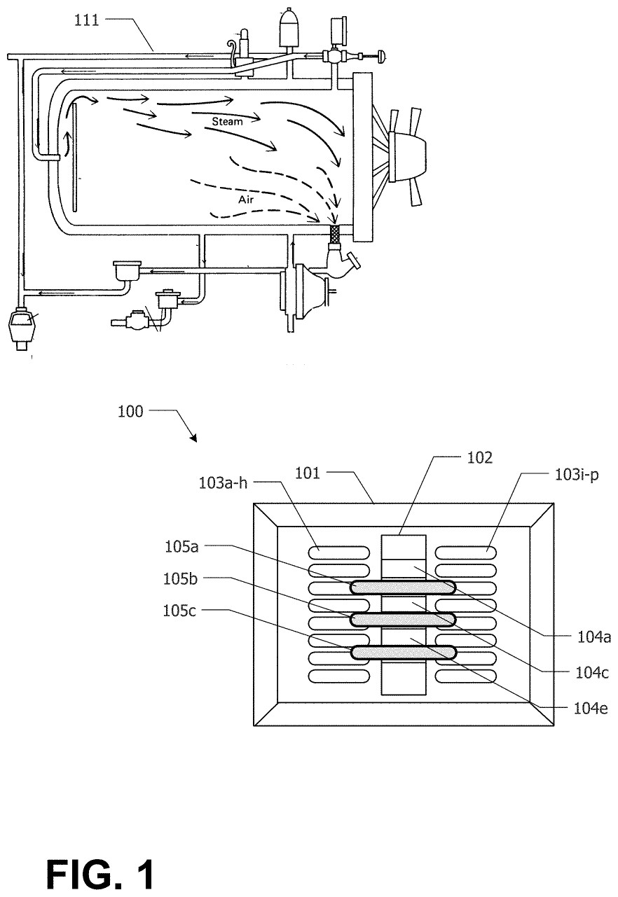

In general, the present disclosure relates to an article of manufacture for providing a cassette to sterilize curing light devices within an autoclave according to the present invention. To better understand the present invention, FIG. 1 illustrates an example embodiment of an article of manufacture for providing a cassette to sterilize curing light devices within an autoclave according to the present invention. A cassette for sterilizing curing light devices 100 is shown in FIG. 1 in which the curing light devices 105a-c may be placed in the cassette before the curing light devices 100 are placed within an autoclave 111 along with other dental instruments for cleaning. The cassette for sterilizing curing light devices 100 comprises an enclosed container 101 having a saddle 102 coupled to an inner bottom surface 112 and a plurality of venting openings 103a-p through the inner bottom surface 112 of the enclosed container 101. Once the curing light devices 105a-c are secured within the saddle 102, the entire cassette for sterilizing curing light devices 100 is inserted into the autoclave 111 for sterilization using the standard autoclave practice.

The saddle 102 within the enclosed container 101 comprises a plurality of device securing locations 104a-e along a top surface of the saddle 102. The curing light devices 105a-c are each placed within the plurality of device securing locations 104a-e before the cassette 100 is placed into the autoclave 111. The saddle 102 may be constructed of any size and shape that matches the curing light devices 105a-c that are to be place therein. The saddle 102 is used to constrain the movement of the curing light devices 105a-c when in the autoclave and subjected to heated steam used to sterilize the items for reuse.

The cassette to sterilize curing light devices 100 may be made of a plastic material that is capable of encountering the temperatures of the steam within the autoclave 111. The cassette to sterilize curing light devices 100 is placed upon a shelf within the autoclave allowing the steam to pass through the cassette 100 and out of its open top during processing of the curing light devices 105a-c.

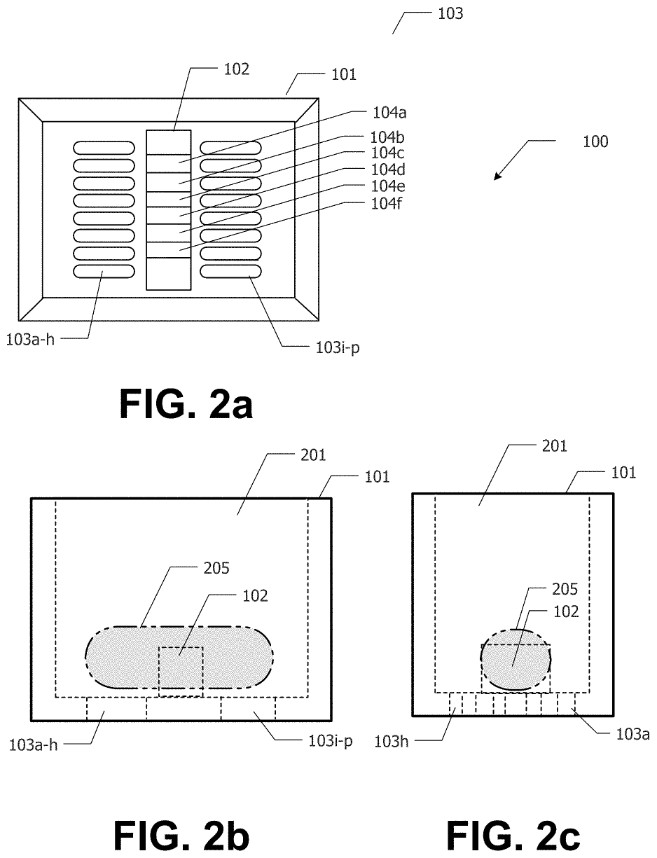

FIGS. 2a-c illustrate multiple views of an article of manufacture for providing a cassette to sterilize curing light devices within an autoclave according to the present invention. FIG. 2a shows a top view of the cassette for sterilizing curing light devices 100. The top view of FIG. 2a is similar to the view disclosed in reference to FIG. 1. The cassette for sterilizing curing light devices 100 has a container 101 with a cavity 201 into which one or more of the curing light devices 205 may be placed. The saddle 102 has a plurality of device securing locations 104a-e along the saddle 102 that accepts the curing light devices 205.

In a preferred embodiment, the cassette for sterilizing curing light devices 100 is made of composition is polythene. The saddle 102 is 7/16 of an inch wide which holds the curing light. The cassette has the capability to hold 3 saddles. The base of the cassette 100 holding the saddles is made of polysulfone. The cassette base 100 has dimensions for a preferred embodiment of a length of 4¼ inches, a width of 3¼ inches, a height of 1/12 ??? inches. and a thickness of ¼ of an inch. The here are 16 venting openings 103a-p have dimensions of 1⅛ inches long by 3/16 inches wide. The saddle 102 may be secured to the base of the cassette 100 using a pair of stainless steel rivets. Other securing devices including screws, nuts and bolts, and related devices may also be used.

FIG. 2b shows a side view of the cassette for sterilizing curing light devices 100. FIG. 2c shows an end view of the cassette for sterilizing curing light devices 100. In the views of FIGS. 2b-c, the curing light devices 205 are shown to be within structures of the saddle 1-2 that have side walls between the plurality of device securing locations 104a-e to keep the devices separated from each other. These side walls 202a-b are along each side of the curing light device 205 in FIGS. 2b-c. While FIGS. 2a-c show a single curing light device 205 within the enclosing container 101, more than one curing light device 205 may be present within the cavity 201 of the enclosing container 101 when the saddle 102 includes multiple securing locations 104a-c.

Even though particular combinations of features are recited in the present application, these combinations are not intended to limit the disclosure of the invention. In fact, many of these features may be combined in ways not specifically recited in this application. In other words, any of the features mentioned in this application may be included in this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed is:

1. An article of manufacture providing a cassette to sterilize curing light devices within an autoclave, the cassette comprises:
an enclosing cassette base having a base surface, sidewalls, and an open top;
a saddle for restraining a curing light device; and
a plurality of venting openings in the base surface.

2. The cassette according to claim 1, wherein the cassette is made of a cassette material permitting use in the autoclave having steam when in operation.

3. The cassette according to claim 2, wherein the cassette material comprises plastic.

4. The cassette according to claim 1, wherein the saddle is made of polythene.

5. The cassette according to claim 4, wherein the saddle is securing to the cassette base surface using a securing device.

6. The cassette according to claim 4, wherein the securing device comprises stainless steel rivets, screws, and nuts and bolt pairs.

7. The cassette according to claim 2, wherein the cassette comprises a plurality of saddles.

8. The cassette according to claim 2, wherein the plurality of saddles comprise multiple individual saddles of different sizes holding different curing light devices.
